(19) United States
(12) Patent Application Publication
(10) Pub. No.: US 2026/0137735 A1
(43) Pub. Date: May 21, 2026

(54) METHOD FOR PROLONGING TELOMERE LENGTH

(71) Applicant: Yu-Lin Yang, Kaohsiung City (TW)

(72) Inventor: Yu-Lin Yang, Kaohsiung City (TW)

(21) Appl. No.: 19/452,537

(22) Filed: Jan. 19, 2026

Related U.S. Application Data
(62) Division of application No. 18/302,922, filed on Apr. 19, 2023.

(51) Int. Cl. A61K 35/745; A61K 35/00; A61K 35/747; A61K 36/287; A61K 36/41; A61K 36/42; A61K 36/48; A61K 36/537; A61K 36/605; A61K 36/73; A61K 36/736; A61P 39/00

(52) U.S. Cl. CPC A61K 35/745; A61K 35/747; A61K 36/287; A61K 36/41; A61K 36/42; A61K 36/48; A61K 36/537; A61K 36/605; A61K 36/73; A61K 36/736; A61P 39/00; A61K 2035/115

(57) ABSTRACT

A method for prolonging telomere length in a subject is disclosed. The method comprises administering a composition including Salvia miltiorrhiza extract, wheat α-amylase inhibitor, and probiotic powder. Administration of the composition prolongs telomere length and provides cellular effects associated with prolonged telomere length, such as a reduction in telomere-associated cellular age.

### CROSS REFERENCE TO RELATED APPLICATION

This is a divisional application of U.S. patent application Ser. No. 18/302,922 filed on Apr. 19, 2023.

### BACKGROUND OF THE INVENTION

### 1. Field of the Invention

The present invention relates to a method for improving cellular health and, more particularly, to a method for prolonging telomere length in a subject by administering a composition with NRF mitochondria activating ingredients.

### 2. Description of the Related Art

Telomeres are repetitive DNA sequences at the end of cell chromosomes. Telomeres function to protect chromosome integrity, to support cell replication, and to influence the cell division cycle. Telomere length is generally considered to be positively correlated with cellular health and viability. Progressive shortening of telomeres is associated with cellular aging.

Methods to influence telomere length include sleep, exercise, and stress management. However, there remains a lack of effective method based on nutritional supplementation that have been demonstrated to prolong telomere length in a statistically significant and measurable manner.

### SUMMARY OF THE INVENTION

In view of the foregoing, the present invention provides a method for prolonging telomere length in a subject, thereby improving cellular conditions associated with telomere length maintenance, such as a reduction in telomere-associated cellular age.

According to the method of the present invention, a composition is administered to the subject, wherein the composition comprises a Salvia miltiorrhiza extract, a wheat α-amylase inhibitor, and probiotic powder which are mixed according to a predetermined weight proportion. The probiotic powder includes Bifidobacterium animalis subsp. lactis and Lactobacillus rhamnosus.

The Salvia miltiorrhiza extract contains NRF mitochondrial activating ingredients, which is extracted and concentrated to provide mitochondrial activation and increased cellular energy availability.

The wheat α-amylase inhibitor interrupts the conversion of starch into sugars and the probiotic powder improves the microbial phase of intestinal tract. Cellular conditions influenced by telomere length are multifactorial. By administering the composition, the method of the present invention prolongs telomere length and provide beneficial cellular effects associated with prolonged telomere length.

### BRIEF DESCRIPTION OF THE DRAWINGS

No drawing is provided.

### DETAILED DESCRIPTION OF THE INVENTION

In accordance with the preferred embodiment of the present invention, a method for prolonging telomere length in a subject is provided by administering a composition with NRF mitochondria activating ingredients, thereby improving cellular conditions associated with telomere length maintenance.

The administered composition comprises a Salvia miltiorrhiza extract with a weight proportion of 1% to 25%, a wheat α-amylase inhibitor with a weight proportion of 5% to 45%, and probiotic powder with a weight proportion of 4% to 30%. The probiotic powder includes Bifidobacterium animalis subsp. lactis with a weight proportion of 50% and Lactobacillus rhamnosus with a weight proportion of 50%.

By administering the composition, the Salvia miltiorrhiza extract contributes to mitochondrial activation, the wheat α-amylase inhibitor contributes to reduce sugar absorption and cut sugar, and the probiotic powder contributes to reduce fat. Preferably, the Salvia miltiorrhiza extract includes tanshinone, salvianolic acid A, salvianolic acid B, Danshensu, salvianolic acid, silymarin, salvianol, salvianoline, and tanshinazine. Without being bound by theory, the observed prolonging telomere length may be attributable to the combined presence of the Salvia miltiorrhiza extract, the wheat α-amylase inhibitor and the probiotic powder, each of which is described herein as contributing different effects. The combined administration of these components may result in overall cellular conditions that are not predictable from any single component alone. Such description is provided to assist in understanding the experimental observations and is not intended to define any specific biological mechanism or to limit the scope of the invention.

In another preferred embodiment of the present invention, the administered composition further comprises an alfalfa extract with a weight proportion of 0.5% to 1%. The alfalfa extract is used to activate mitochondria as well.

In another preferred embodiment of the present invention, the administered composition further comprises a Momordica charantia extract, a Fructus mume extract, a strawberry extract, a Rhodiola rosea extract, a Chrysanthemum extract, and a mulberry leaf extract with a weight proportion of 1% to 2%. The Momordica charantia extract, the Fructus mume extract, the strawberry extract, the Rhodiola rosea extract, the Chrysanthemum extract, and the mulberry leaf extract are used to reduce sugar and cut sugar.

Referring to TABLE 1, in an experimental example, 20 subjects administered the composition at a dosage of 4 grams per day for two months. Oral mucosal smears were collected to extract cellular DNA, and telomere length was measured by QPCR before and after administration.

TABLE 1

| | telemore | telemore | telomere | | | telomere |
|---|---|---|---|---|---|---|
| age of | length | length | length | telemore | telemore | age |
| subjects | (before) | (after) | variation (Kb) | age (before) | age (after) | variation |
| 52 | 1.8 | 1.9 | 0.1 | 80 | 79 | −1 |
| 37 | 3.1 | 3.4 | 0.3 | 68 | 65 | −3 |
| 35 | 3.1 | 4.5 | 1.4 | 68 | 54 | −14 |
| 67 | 6.1 | 7.4 | 1.3 | 38 | 25 | −13 |
| 51 | 4.5 | 5.9 | 1.4 | 54 | 40 | −14 |
| 51 | 6.5 | 7.0 | 0.5 | 34 | 30 | −4 |
| 47 | 4.3 | 5.1 | 0.8 | 56 | 48 | −8 |
| 34 | 5.3 | 8.1 | 2.8 | 46 | 18 | −28 |
| 65 | 2.5 | 6.9 | 4.4 | 74 | 30 | −44 |
| 44 | 4.2 | 5.0 | 0.8 | 57 | 49 | −8 |
| 55 | 2.5 | 2.9 | 0.4 | 74 | 70 | −4 |
| 40 | 3.5 | 3.4 | −0.1 | 64 | 65 | 1 |
| 35 | 3.4 | 4.5 | 1.1 | 65 | 54 | −11 |
| 65 | 3.5 | 3.9 | 0.4 | 64 | 60 | −4 |
| 28 | 8.1 | 8.2 | 0.1 | 19 | 18 | −1 |
| 48 | 4.4 | 4.4 | 0.0 | 55 | 55 | 0 |
| 45 | 5.7 | 7.8 | 2.1 | 42 | 22 | −20 |
| 46 | 4.5 | 4.0 | −0.5 | 54 | 59 | 5 |
| 32 | 5.1 | 8.1 | 3.0 | 48 | 18 | −30 |
| 60 | 4.0 | 5.1 | 1.1 | 59 | 48 | 11 |

The experimental results show that telomere length of the subjects was prolonged by an average of 1.074 Kb (kilo base pairs), demonstrating that the method of the present invention effectively prolongs telomere length and provides beneficial cellular effects associated with prolonged telomere length such as a reduction in telomere-associated cellular age as reflected by changes in measured telomere length.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

What is claimed is:

1. A method for prolonging telomere length in a subject, comprising administering to the subject a composition effective to prolong the telomere length of the subject,
with the composition comprising:
a Salvia miltiorrhiza extract with a weight proportion of 1% to 25%, a wheat α-amylase inhibitor with a weight proportion of 5% to 45%, and probiotic powder with a weight proportion of 4% to 30%;
wherein:
the probiotic powder includes Bifidobacterium animalis subsp. lactis with a weight proportion of 50% and Lactobacillus rhamnosus with a weight proportion of 50%.

2. The method as claimed in claim 1, wherein the composition further comprises an alfalfa extract with a weight proportion of 0.5% to 1%.

3. The method as claimed in claim 1, wherein the composition further comprises a Momordica charantia extract, a Fructus mume extract, a strawberry extract, a Rhodiola rosea extract, a Chrysanthemum extract, and a mulberry leaf extract with a weight proportion of 1% to 2%.
